(19) United States
(12) Patent Application Publication
(10) Pub. No.: US 2024/0339187 A1
(43) Pub. Date: Oct. 10, 2024

(54) MEDICAL SUPPORT SYSTEM, REPORT CREATION SUPPORT METHOD, AND INFORMATION PROCESSING APPARATUS

(71) Applicant: OLYMPUS MEDICAL SYSTEMS CORP., Tokyo (JP)

(72) Inventors: Haruhiko SAKAYORI, Tokyo (JP); Yuta KOBAYASHI, Tokyo (JP)

(73) Assignee: OLYMPUS MEDICAL SYSTEMS CORP., Tokyo (JP)

(21) Appl. No.: 18/746,897

(22) Filed: Jun. 18, 2024

Related U.S. Application Data
(63) Continuation of application No. PCT/JP2022/001462, filed on Jan. 17, 2022.

(51) Int. Cl. G16H 15/00; G06T 7/00; G06T 7/55; G06T 7/60

(52) U.S. Cl. CPC G16H 15/00; G06T 7/0014; G06T 7/55; G06T 7/60; G06T 2200/24; G06T 2207/20092; G06T 2207/30096

(57) ABSTRACT

An image acquisition unit acquires a user-captured image captured through a capture operation by a user and a computer-captured image captured by a computer including a lesion. An image specifying unit specifies computer-captured images that include a lesion not included in the user-captured images as candidate images for attachment. A display screen generation unit generates a selection screen that is for selecting an image to be attached to a report and that includes the user-captured images and the candidate images for attachment and displays the selection screen on a display device.

### CROSS-REFERENCE TO RELATED APPLICATIONS

This application is based upon and claims the benefit of priority from the International Application No. PCT/JP2022/001462, filed on Jan. 17, 2022, the entire contents of which are incorporated.

### BACKGROUND

### 1. Technical Field

The present disclosure relates to a medical support system, an information processing apparatus, and a report creation support method for supporting report creation.

### 2. Description of the Related Art

In endoscopic examination, a doctor observes endoscopic images displayed on a display device and, upon finding a lesion, operates an endoscope release switch to capture (save) endoscopic images of the lesion. After the examination is completed, the doctor creates a report by inputting examination results on a report input screen and selecting endoscopic images to be attached to the report from the multiple captured endoscopic images. JP 2017-86274A discloses a report input screen that displays a list of the multiple captured endoscopic images as candidate images for attachment.

In the related art, endoscopic images displayed in a list on a report input screen are limited to images captured by a capturing operation (release switch operation) performed by a doctor. Therefore, while a doctor performs an endoscopic examination in a short period of time so as not to burden the patient, it is not possible to attach images forgotten to be captured by the doctor to a report. Using a computer-aided diagnosis (CAD) system that has been researched in recent years, it is also possible to display images detected by the CAD system as candidate images for attachment on a report input screen. However, if there is a large number of images in which the CAD system has detected lesions, the time and effort required for the doctor to select images to be attached to a report on the report input screen will increase.

### SUMMARY

The present disclosure has been made in view of the aforementioned problems and a general purpose thereof is to provide a medical support technology that allows images captured by a computer such as a CAD system to be efficiently displayed.

A medical support system according to one embodiment of the present disclosure includes: one or more processors comprising hardware. The one or more processors are configured to: acquire a first image captured through a capture operation by a user and a computer-captured image captured by a computer including a lesion; specify the computer-captured image including a lesion not included in the first image as a second image; and generate a selection screen that is for selecting an image to be attached to a report and that includes the first image and the second image.

A report creation support method according to another embodiment of the present disclosure includes: acquiring a first image captured through a capture operation by a user; acquiring a computer-captured image captured by a computer including a lesion; specifying the computer-captured image including a lesion not included in the first image as a second image; and generating a selection screen that is for selecting an image to be attached to a report and that includes the first image and the second image.

An information processing apparatus according to yet another embodiment of the present disclosure includes: one or more processors comprising hardware, wherein the one or more processors are configured to: acquire a first image captured through a capture operation by a user and a computer-captured image captured by a computer including a lesion; specify the computer-captured image including a lesion not included in the first image as a second image; and generate a selection screen that is for selecting an image to be attached to a report and that includes the first image and the second image.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing the configuration of a medical support system according to an embodiment;

FIG. 2 is a diagram showing functional blocks of a server device;

FIG. 3 is a diagram showing examples of information linked to captured images;

FIG. 4 is a diagram showing functional blocks of an information processor;

FIG. 5 is a diagram showing an example of a selection screen for selecting endoscopic images;

FIG. 6 is a diagram showing an example of a report creation screen for inputting examination results;

FIG. 7 is a diagram showing a table stored in a 5 priority memory unit;

FIG. 8 is a diagram showing another example of the selection screen for selecting endoscopic images;

FIG. 9 is a diagram showing an example of the selection screen when all computer-captured images are displayed; and

FIG. 10 is a diagram showing other examples of information linked to captured images.

### DETAILED DESCRIPTION

The disclosure will now be described by reference to the preferred embodiments. This does not intend to limit the scope of the present disclosure, but to exemplify the disclosure.

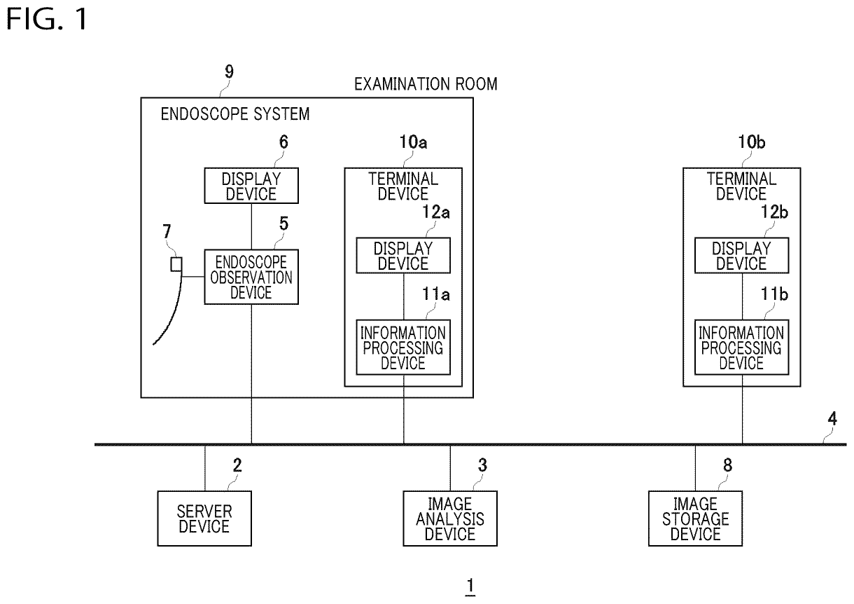

FIG. 1 shows the configuration of a medical support system 1 according to an embodiment. The medical support system 1 is provided in a medical facility such as a hospital where endoscopic examinations are performed. In the medical support system 1, a server device 2, an image analysis device 3, an image storage device 8, an endoscope system 9, and a terminal device 10b are communicably connected via a network 4 such as a local area network (LAN). The endoscope system 9 is installed in an examination room and has an endoscope observation device 5 and a terminal device 10a. In the medical support system 1, the server device 2, the image analysis device 3, and the image storage device 8 may be provided outside the medical facility, for example, in a cloud server.

The endoscope observation device 5 is connected to an endoscope 7 to be inserted into the digestive tract of a patient. The endoscope 7 has a light guide for illuminating the inside of the digestive tract by transmitting illumination light supplied from the endoscope observation device 5, and the distal end of the endoscope 7 is provided with an illumination window for emitting the illumination light transmitted by the light guide to living tissue and an image-capturing unit for image-capturing the living tissue at a predetermined cycle and outputting an image-capturing signal to the endoscope observation device 5. The image-capturing unit includes a solid-state imaging device, e.g., a CCD image sensor or a CMOS image sensor, that converts incident light into an electric signal.

The endoscope observation device 5 performs image processing on the image-capturing signal photoelectrically converted by a solid-state imaging device of the endoscope 7 so as to generate an endoscopic image and displays the endoscopic image on the display device 6 in real time. In addition to normal image processing such as A/D conversion and noise removal, the endoscope observation device 5 may include a function of performing special image processing for the purpose of highlighting, etc. The endoscope observation device 5 generates endoscopic images at a predetermined cycle, e.g., 1/60 seconds. The endoscope observation device 5 may be formed by one or more processors with dedicated hardware or may be formed by one or more processors with general-purpose hardware.

According to the examination procedure, the doctor observes an endoscopic image displayed on the display device 6. The doctor observes the endoscopic image while moving the endoscope 7, and the doctor operates the release switch of the endoscope 7 when a lesion appears on the display device 6. When the release switch is operated, the endoscope observation device 5 captures (saves) an endoscopic image at the time when the release switch is operated and then transmits the captured endoscopic image to the image storage device 8 along with information identifying the endoscopic image (image ID). The endoscope observation device 5 may transmit a plurality of captured endoscopic images all at once to the image storage device 8 after the examination is completed. The image storage device 8 records the endoscopic images transmitted from the endoscope observation device 5 in association with an examination ID for identifying the endoscopic examination. The endoscopic images stored in the image storage device 8 are used by the doctor to create an examination report.

The terminal device 10a is installed in the examination room with an information processing device 11a and a display device 12a. The terminal device 10a is used by doctors, nurses, and others in order to check information on lesions in real time during endoscopic examinations. The information processing device 11a acquires information on a lesion from the server device 2 and/or the image analysis device 3 during an endoscopic examination and displays the information on the display device 12a. For example, the display device 12a may display the size of the lesion, the depth of the lesion, and the qualitative diagnostic results of the lesion as analyzed by the image analysis device 3.

The terminal device 10b is installed in a room other than the examination room with an information processing device 11b and a display device 12b. The terminal device 10b is used when a doctor creates a report of an endoscopic examination. The terminal devices 10a and 10b are formed with one or more processors having general-purpose hardware.

In the medical support system 1 according to the embodiment, the endoscope observation device 5 displays endoscopic images in real time through the display device 6, and provides the endoscopic images along with meta information of the images to the image analysis device 3 in real time. The meta information includes at least the frame number and imaging time information of each image. The frame number is information indicating the number of the frame after the endoscope 7 starts imaging. In other words, the frame number may be a serial number indicating the order of image-capturing. For example, the frame number of an endoscopic image captured first is set to “1,” and the frame number of an endoscopic image captured second is set to “2.”

The image analysis device 3 is an electronic calculator (computer) that analyzes endoscopic images to detect lesions in the endoscopic images and performs qualitative diagnosis of the detected lesions. The image analysis device 3 may be a computer-aided diagnosis (CAD) system with an artificial intelligence (AI) diagnostic function. The image analysis device 3 may be formed by one or more processors with dedicated hardware or may be formed by one or more processors with general-purpose hardware.

The image analysis device 3 may use a trained model that is generated by machine learning using endoscopic images for learning and information concerning a lesion area contained in the endoscopic images as training data. Annotation work on the endoscopic images is performed by annotators with expertise, such as doctors, and machine learning may use CNN, RNN, LSTM, etc., which are types of deep learning. Upon input of an endoscopic image, this trained model outputs information indicating an imaged organ, information indicating an imaged site, and information concerning an imaged lesion (lesion information). The lesion information output by the image analysis device 3 includes at least information on the presence or absence of a lesion indicating whether the endoscopic image contains a lesion or not. When the lesion is contained, the lesion information may include information indicating the size of the lesion, information indicating the location of the outline of the lesion, information indicating the shape of the lesion, information indicating the invasion depth of the lesion, and a qualitative diagnosis result of the lesion. The qualitative diagnostic result of the lesion includes the type of lesion. During an endoscopic examination, the image analysis device 3 is provided with endoscopic images from the endoscope observation device 5 in real time and outputs information indicating the organ, information indicating the site, and lesion information for each endoscopic image. Hereafter, the information indicating the organ, the information indicating the site, and the lesion information are collectively referred to as “image meta information.”

The image analysis device 3 according to the embodiment has a function of measuring the amount of time spent observing a lesion by the doctor (hereinafter also referred to as the “user”). In a case where the user operates the release switch to capture an endoscopic image, the image analysis device 3 measures the time from when a lesion included in the endoscopic image is first included in a previous endoscopic image until the release switch is operated as the time during which the lesion has been observed by the user. In other words, the image analysis device 3 specifies the time from when the lesion is first imaged until the user operates a capture operation (release switch operation) as the observation time of the lesion. In the embodiment, “imaging” refers to operation of converting incident light into an electrical signal performed by a solid-state image sensor of an endoscope 7, and “capturing” refers to operation of saving (recording) an endoscopic image generated by the endoscope observation device 5.

When the user performs a capture operation, the endoscope observation device 5 provides the frame number, imaging time, and image ID of the captured endoscopic image to the image analysis device 3, along with information indicating that the capture operation has been performed (capture operation information). Upon acquiring the capture operation information, the image analysis device 3 specifies the observation time of the lesion and provides the image ID, the frame number, the imaging time information, the observation time of the lesion, and image meta information of the provided frame number to the server device 2. The server device 2 records the frame number, the imaging time information, the observation time of the lesion, and the image meta information in association with the image ID of the endoscopic image.

The image analysis device 3 according to the embodiment has a function of automatically capturing the endoscopic image when detecting a lesion in the endoscopic image. When the same lesion is included in a 10-second endoscopic video, the image analysis device 3 may automatically capture an endoscopic image in which the lesion is first detected, and may not automatically capture the lesion even when the same lesion is detected in subsequent endoscopic images. The image analysis device 3 only needs to capture one endoscopic image that includes the detected lesion in the end. For example, after capturing multiple endoscopic images that include the same lesion, the image analysis device 3 may select one endoscopic image in which the lesion is most clearly imaged and discard the other captured endoscopic images. In order to clarify the subject of capturing, endoscopic images captured through a capture operation by the user may be referred to as “user-captured images,” and endoscopic images automatically captured by the image analysis device 3 may be referred to as “computer-captured images.”

Upon acquiring a computer-captured image, the image analysis device 3 specifies the observation time of the lesion included in the computer-captured image ex post. The image analysis device 3 may specify the time during which the lesion has been captured as the observation time of the lesion. For example, if the lesion is included in a 10-second endoscopic video, i.e., if the lesion has been imaged for 10 seconds and displayed on the display device 12a, the image analysis device 3 may specify the observation time of the lesion as 10 seconds. Therefore, the image analysis device 3 measures the time from when the lesion comes into the frame and the imaging of the lesion starts until the lesion goes out of the frame and the imaging of the lesion ends, and specifies the time as the observation time of the lesion. The image analysis device 3 provides the computer-captured image to the server device 2 along with the frame number, imaging time information, observation time of the lesion, and image meta information of the computer-captured image.

When the user finishes the endoscopic examination, the user operates an examination completion button on the endoscope observation device 5. The operation information of the examination completion button is provided to the server device 2 and the image analysis device 3, and the server device 2 and the image analysis device 3 recognize the completion of the endoscopic examination.

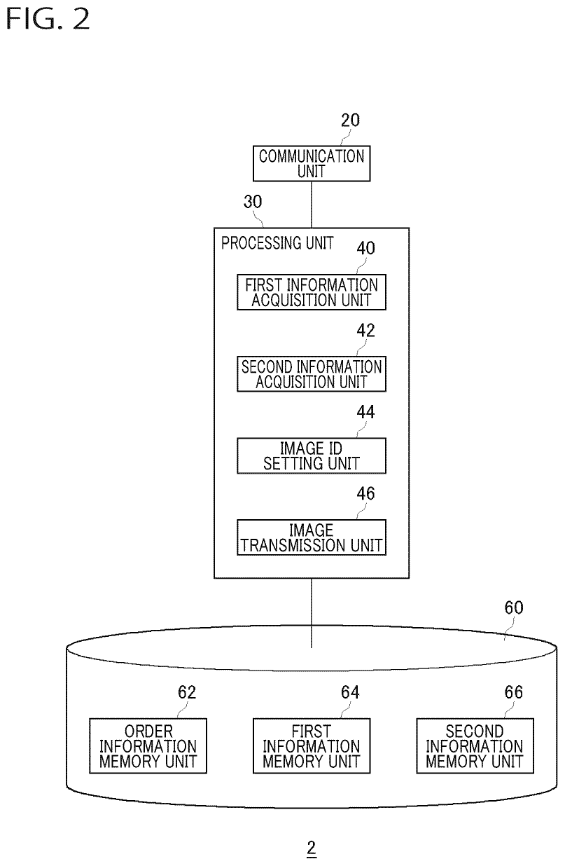

FIG. 2 shows functional blocks of the server device 2. The server device 2 includes a communication unit 20, a processing unit 30, and a memory device 60. The communication unit 20 transmits and receives information such as data and instructions between the image analysis device 3, the endoscope observation device 5, the image storage device 8, the terminal device 10a, and the terminal device 10b via the network 4. The processing unit 30 includes a first information acquisition unit 40, a second information acquisition unit 42, an image ID setting unit 44, and an image transmission unit 46. The memory device 60 has an order information memory unit 62, a first information memory unit 64, and a second information memory unit 66. The order information memory unit 62 stores information for endoscopic examination orders.

The server device 2 includes a computer. Various functions shown in FIG. 2 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 2 are realized by cooperation between hardware and software. Therefore, a person skilled in the art should appreciate that there are many ways of accomplishing these functional blocks in various forms in accordance with the components of hardware only, software only, or the combination of both.

The first information acquisition unit 40 acquires the image ID, frame number, imaging time information, observation time, and image meta information of a user-captured image from the image analysis device 3 and stores these pieces of information in the first information memory unit 64 along with information indicating that the image is a user-captured image. For example, if a user performs seven capture operations during an endoscopic examination, the first information acquisition unit 40 acquires the respective image IDs, frame numbers, imaging time information, observation time, and image meta information of seven user-captured images from the image analysis device 3 and stores these pieces of information in the first information memory unit 64 along with information indicating that the images are user-captured images. The first information memory unit 64 may store the information indicating that the images are user-captured images, the frame numbers, the imaging time information, the observation time, and the image meta information in association with the respective image IDs. Image IDs are assigned to user-captured images by the endoscope observation device 5, and the endoscope observation device 5 assigns the image IDs in order of the imaging time, starting from 1. Therefore, image IDs 1 to 7 are assigned to the seven user-captured images, respectively, in this case.

The second information acquisition unit 42 acquires computer-captured images and the respective frame numbers, imaging time information, observation time, and image meta information of the computer-captured images from the image analysis device 3 and stores these pieces of information in the second information memory unit 66 along with information indicating that the images are computer-captured images. At this point, since the computer-captured images have not been assigned image IDs, the image ID setting unit 44 may set image IDs for the computer-captured images according to the imaging time. More specifically, the image ID setting unit 44 sets the image IDs of the computer-captured images such that the image IDs are not the same as the image IDs of the user-captured images. When image IDs 1 to 7 are assigned to the seven user-captured images as described above, the image ID setting unit 44 may assign image IDs to the computer-captured images in the order of imaging time, starting from 8. If the image analysis device 3 has performed automatic capturing for seven times, i.e., a total of seven lesions have been detected in an endoscopic examination and seven endoscopic images have been automatically captured, the image ID setting unit 44 may set the image IDs starting from 8 in order of the imaging time starting from the earliest time. Thus, image IDs 8 to 14 are set for the seven computer-captured images in this case. The second information memory unit 66 stores the information indicating that the images are computer-captured images, the frame numbers, the imaging time information, the observation time, and the image meta information in association with the respective image IDs.

The image transmission unit 46 transmits the computer-captured images along with the assigned image IDs to the image storage device 8. This causes the image storage device 8 to store both the user-captured images with the image IDs 1 to 7 and the computer-captured images with the image IDs 8 to 14 captured during the endoscopic examination.

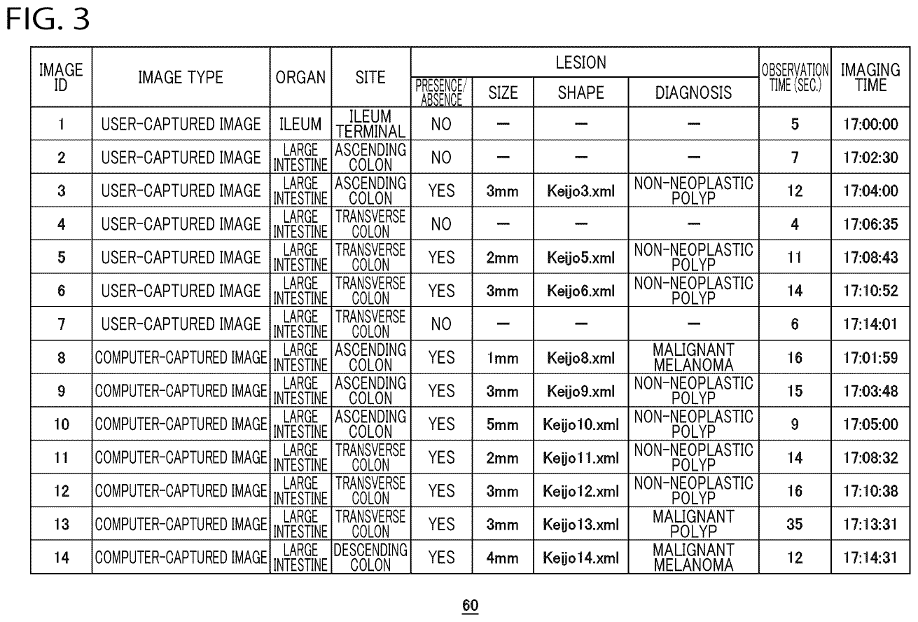

FIG. 3 shows examples of information linked to the captured images. Information on the user-captured images with the image IDs 1 to 7 is stored in the first information memory unit 64, and information on the computer-captured images with the image IDs 8 to 14 is stored in the second information memory unit 66.

Information indicating whether the image is a user-captured image or a computer-captured image is stored in an “IMAGE TYPE” field. An “ORGAN” field stores information indicating the organ included in the image, i.e., the organ that has been imaged, and a “SITE” field stores information indicating the site of the organ included in the image, i.e., the site of the organ that has been imaged.

A “PRESENCE/ABSENCE” field stores information indicating whether or not a lesion has been detected by the image analysis device 3 in the lesion information. Since all computer-captured images include a lesion, “YES” is stored in the “PRESENCE/ABSENCE” field for the computer-captured images. A “SIZE” field stores information indicating the longest diameter of the bottom surface of the lesion, a “SHAPE” field stores coordinate information expressing the contour shape of the lesion, and a “DIAGNOSIS” field stores the qualitative diagnosis result of the lesion. An “OBSERVATION TIME” field stores the observation time derived by the image analysis device 3. An “IMAGING TIME” field stores information indicating the imaging time of the image. The “IMAGING TIME” field may include a frame number.

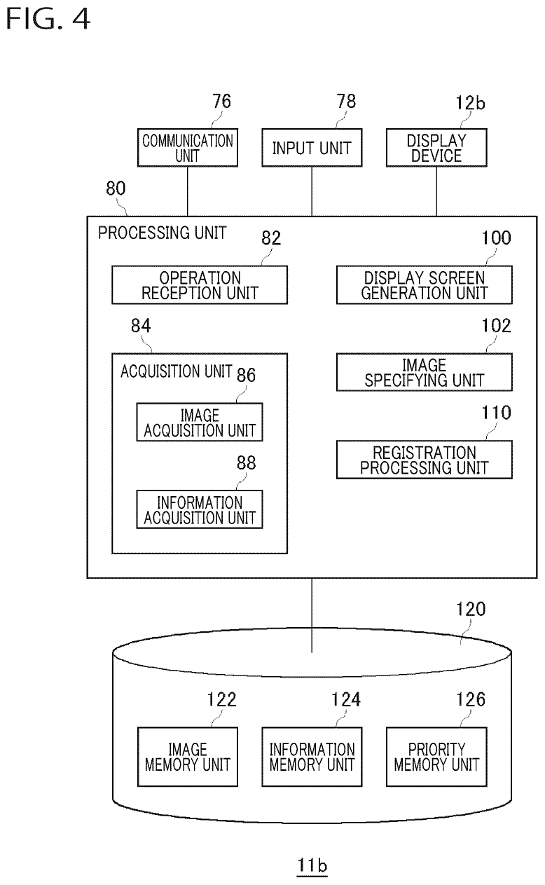

FIG. 4 shows the functional blocks of the information processing device 11b. The information processing device 11b has the function of selecting endoscopic images to be displayed on the report input screen and includes a communication unit 76, an input unit 78, a processing unit 80, and a memory device 120. The communication unit 76 transmits and receives information such as data and instructions between the server device 2, the image analysis device 3, the endoscope observation device 5, the image storage device 8, and the terminal device 10a via the network 4. The processing unit 80 includes an operation reception unit 82, an acquisition unit 84, a display screen generation unit 100, an image specifying unit 102, and a registration processing unit 110, and the acquisition unit 84 has an image acquisition unit 86 and an information acquisition unit 88. The memory device 120 has an image memory unit 122, an information memory unit 124, and a priority memory unit 126.

The information processing device 11b includes a computer. Various functions shown in FIG. 4 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 4 are realized by cooperation between hardware and software. Therefore, a person skilled in the art should appreciate that there are many ways of accomplishing these functional blocks in various forms in accordance with the components of hardware only, software only, or the combination of both.

After the completion of an endoscopic examination, the user, a doctor, inputs a user ID and a password to the information processing device 11b so as to log in. An application for preparing an examination report is activated when the user logs in, and a list of already performed examinations is displayed on the display device 12b. The list of already performed examinations displays examination information such as a patient name, a patient ID, examination date and time, examination, and the like in a list, and the user operates the input unit 78 such as a mouse or a keyboard so as to select an examination for which a report is to be created. When the operation reception unit 82 receives an examination selection operation, the image acquisition unit 86 acquires a plurality of endoscopic images linked to the examination ID of the selected examination from the image storage device 8 and stores the endoscopic images in the image memory unit 122.

In the embodiment, the image acquisition unit 86 acquires the user-captured images with the image IDs 1 to 7 captured through capture operations by the user and the computer-captured images including a lesion with the image IDs 8 to 14 captured by the image analysis device 3, and stores the images in the image memory unit 122. The display screen generation unit 100 generates a selection screen for selecting endoscopic images to be attached to the report and displays the selection screen on the display device 12b.

As described above, there are as many computer-captured images as the number of lesions detected by the image analysis device 3. In the embodiment, seven endoscopic images are automatically captured by the image analysis device 3. However, depending on the endoscopic examination, it is also assumed that the image analysis device 3 may detect tens to hundreds of lesions and automatically capture tens to hundreds of endoscopic images. In such a case, displaying all the automatically captured endoscopic images on the selection screen requires a lot of effort for the user to make selections, which is not preferable. Therefore, in the embodiment, the image specifying unit 102 narrows down the computer-captured images to be displayed on the selection screen from among a plurality of computer-captured images. Hereinafter, the computer-captured images to be displayed on the selection screen are referred to as “candidate images for attachment.”

The information acquisition unit 88 acquires information associated with the captured images from the memory device 60 of the server device 2 and stores the information in the information memory unit 124. The image specifying unit 102 refers to the information linked to the captured images so as to specify computer-captured images that include a lesion not included in the user-captured images as “candidate images for attachment.” By having the image specifying unit 102 specifies the computer-captured images that include a lesion not included in the user-captured images as candidate images for attachment, computer-captured images that include the same lesion included in the user-captured images are not displayed on the selection screen in duplicate.

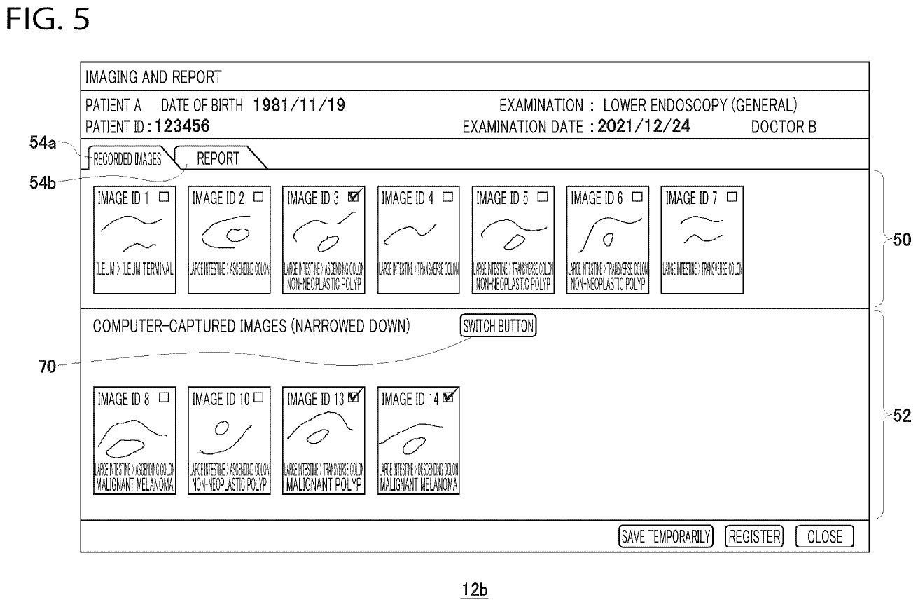

FIG. 5 shows an example of the selection screen for selecting endoscopic images to be attached to a report. The selection screen forms part of a report input screen. The selection screen for endoscopic images is displayed on the display device 12b while a recorded image tab 54a is being selected. In the upper part of the selection screen, information such as a patient name, a patient ID, the date of birth, examination, an examination date, and a performing doctor is displayed. These pieces of information are included in the examination order information and may be acquired from the server device 2.

The display screen generation unit 100 generates a selection screen including user-captured images and computer-captured images (candidate images for attachment) that have been narrowed down and displays the selection screen on the display device 12b. Since lesions included in the computer-captured images (candidate images for attachment) are not the same as lesions included in the user-captured images, the user can efficiently select images to be attached to the report.

The display screen generation unit 100 generates a selection screen in which a first region 50 for displaying user-captured images and a second region 52 for displaying computer-captured images (candidate images for attachment) are provided separately and displays the selection screen on the display device 12b. The display screen generation unit 100 displays the user-captured images with the image IDs 1 to 7 in the first region 50, arranged in order according to the order of imaging and the computer-captured images with the image IDs 8, 10, 13, and 14 in the second region 52 in order according to the order of imaging. Displaying the user-captured images and the candidate images for attachment on the same screen by the display device 12b allows the user to efficiently select images to be attached to the report.

A process of narrowing down computer-captured images will be explained in the following. The image specifying unit 102 specifies computer-captured images that include a lesion not included in the user-captured images as “candidate images for attachment” based on information on captured images stored in the information memory unit 124 (see FIG. 3). The image specifying unit 102 may determine whether there are user-captured images that include the same organ as the organ included in the computer-captured images based on information indicating an organ included in the user-captured images with the image IDs 1 to 7 and information indicating an organ included in the computer-captured images, and specify, when there is no user-captured image that includes the same organ as the organ included in the computer-captured images, the computer-captured images as candidate images for attachment. When there is no user-captured image that includes the same organ as the organ included in the computer-captured images, since it is certain that the lesion included in the computer-captured images is not included in the user-captured images, the image specifying unit 102 may specify the computer-captured images as candidate images for attachment.

The image specifying unit 102 may determine whether there are user-captured images that include the same site as the site included in the computer-captured images based on information indicating the site of an organ included in the user-captured images with the image IDs 1 to 7 and information indicating the site of an organ included in the computer-captured images, and specify, when there is no user-captured image that includes the same site as the site included in the computer-captured images, the computer-captured images as candidate images for attachment. When there is no user-captured image that includes the same site as the site included in the computer-captured images, since it is certain that the lesion included in the computer-captured images is not included in the user-captured images, the image specifying unit 102 may specify the computer-captured images as candidate images for attachment.

Based on information indicating the size of the lesion included in the user-captured images with the image IDs 1 to 7 and information indicating the size of the lesion included in the computer-captured images, the image specifying unit 102 determines whether there are user-captured images that include a lesion whose size is the same as or whose difference is within a predetermined range from the size of the lesion included in the computer-captured images. That is, the image specifying unit 102 determines whether or not there are user-captured images that include the lesion whose size is the same as the size of the lesion included in the computer-captured images or whose difference in size from the lesion included in the computer-captured images is within the predetermined range. When there is no user-captured image that includes a lesion whose size is the same as or whose size difference is within the predetermined range from that of the lesion included in the computer-captured images, the image specifying unit 102 may specify the computer-captured images as candidate images for attachment. When there is no user-captured image that includes a lesion whose size is the same as or whose size difference is within the predetermined range from that of the lesion included in the computer-captured images, since it is certain that the lesion included in the computer-captured images is not included in the user-captured images, the image specifying unit 102 may specify the computer-captured images as candidate images for attachment. The predetermined range of the size difference described above may be set as appropriate based on the endoscope used, the type of lesion, the organ to be observed, or the quality of the endoscopic image. For example, if the ratio of the size of the lesion included in the user-captured images to the size of the lesion included in the computer-captured images is a value between 0.8 and 1.2, the image specifying unit 102 may determine that the difference between the size of the lesion included in the computer-captured images and the size of the lesion included in the user-captured images is within the predetermined range. The image specifying unit 102 may specify the size of a lesion based on the area on an image. Based on information indicating the shape of the lesion included in the user-captured images with the image IDs 1 to 7 and information indicating the shape of the lesion included in the computer-captured images, the image specifying unit 102 determines whether there are user-captured images that include a lesion whose shape is the same as or whose difference is within a predetermined range from the shape of the lesion included in the computer-captured images. That is, the image specifying unit 102 determines whether or not there are user-captured images that include the lesion whose shape is the same as the shape of the lesion included in the computer-captured images or whose difference in shape from the lesion included in the computer-captured images is within the predetermined range. When there is no user-captured image that includes a lesion whose shape is the same as or whose shape difference is within the predetermined range from that of the lesion included in the computer-captured images, the image specifying unit 102 may specify the computer-captured images as candidate images for attachment. When there is no user-captured image that includes a lesion whose shape is the same as or whose shape difference is within the predetermined range from that of the lesion included in the computer-captured images, since it is certain that the lesion included in the computer-captured images is not included in the user-captured images, the image specifying unit 102 may specify the computer-captured images as candidate images for attachment. The predetermined range of the shape difference described above may be set as appropriate based on the endoscope used, the type of lesion, the organ to be observed, or the quality of the endoscopic image. Using a known shape similarity determination method, if the value of the similarity between the shape of the lesion included in the user-captured images and the shape of the lesion included in the computer-captured images is a predetermined value (e.g., 80 percent) or more, the image specifying unit 102 may determine that the difference between the shape of the lesion included in the computer-captured images and the shape of the lesion included in the user-captured images is within the predetermined range. As the shape similarity determination method, a method using the contour of a lesion may be employed, or other methods may be employed.

Based on information indicating the type of the lesion included in the user-captured images with the image IDs 1 to 7 and information indicating the type of the lesion included in the computer-captured images, the image specifying unit 102 determines whether there are user-captured images that include a lesion whose type is substantially the same as that of the lesion included in the computer-captured images. When there is no user-captured image that includes a lesion whose type is substantially the same as that of the lesion included in the computer-captured images, the image specifying unit 102 may specify the computer-captured images as candidate images for attachment. When there is no user-captured image that includes the same type of a lesion as the type of the lesion included in the computer-captured images, since it is certain that the lesion included in the computer-captured images is not included in the user-captured images, the image specifying unit 102 may specify the computer-captured images as candidate images for attachment.

The image specifying unit 102 may determine whether there are user-captured images that include an organ, a site, and a lesion that are substantially the same as those in the computer-captured images based on information indicating the organ, information indicating the site, information indicating the size of the lesion, information indicating the shape of the lesion, and information indicating the type of the lesion that are included in the user-captured images with the image IDs 1 to 7 and on information indicating the organ, information indicating the site, information indicating the size of the lesion, information indicating the shape of the lesion, and information indicating the type of the lesion that are included in the computer-captured images. When there is no user-captured image that includes an organ, a site, and a lesion that are substantially the same as those in the computer-captured images, the image specifying unit 102 may specify the computer-captured images as candidate images for attachment. When there is no user-captured image that includes an organ, a site, and a lesion that are substantially the same as those included in the computer-captured images, since it is certain that the lesion included in the computer-captured images is not included in the user-captured images, the image specifying unit 102 may specify the computer-captured images as candidate images for attachment.

The image specifying unit 102 specifies computer-captured images that include a lesion not included in the user-captured images with the image IDs 1 to 7 as candidate images for attachment as described above. In reference to FIG. 3, the image specifying unit 102 identifies that the lesion included in the computer-captured image with the image ID 9 and the lesion included in the user-captured image with the image ID 3 are identical, the lesion included in the computer-captured image with the image ID 11 and the lesion included in the user-captured image with the image ID 5 are identical, and the lesion included in the computer-captured image with the image ID 12 and the lesion included in the user-captured image with the image ID 6 are identical. Therefore, the image specifying unit 102 determines not to include the computer-captured images with the image IDs 9, 11, and 12 in the selection screen and determines the computer-captured images of the image IDs 8, 10, 13, and 14 as candidate images for attachment. After the above narrowing down is performed, the display screen generation unit 100 displays the user-captured images with the image IDs 1 to 7 in the first region 50 and the computer-captured images with the image IDs 8, 10, 13, and 14 in the second region 52.

Each endoscopic image that is displayed on the selection screen is provided with a check box, and the endoscopic image is selected as an attached image of the report when the user operates a mouse to place a mouse pointer on the check box followed by right-clicking. On the selection screen, the operation reception unit 82 receives a user's operation of selecting a user-captured image or a candidate image for attachment. An endoscopic image can be displayed in an enlarged manner when the user places the mouse pointer on the endoscopic image followed by right-clicking, and the user may determine whether to attach the endoscopic image to the report while looking at the endoscopic image displayed in an enlarged manner.

In the example shown in FIG. 5, check marks are displayed in the check boxes of the user-captured image with the image ID 3 and the computer-captured images with the image IDs 13 and 14, indicating that the images are being selected. The registration processing unit 110 temporarily registers the selected endoscopic images with the image IDs 3, 13, and 14 in the image memory unit 122 as report attached images when the user operates a temporarily save button using the input unit 78. After selecting the attached images, the user selects a report tab 54b to display a report input screen on the display device 12b.

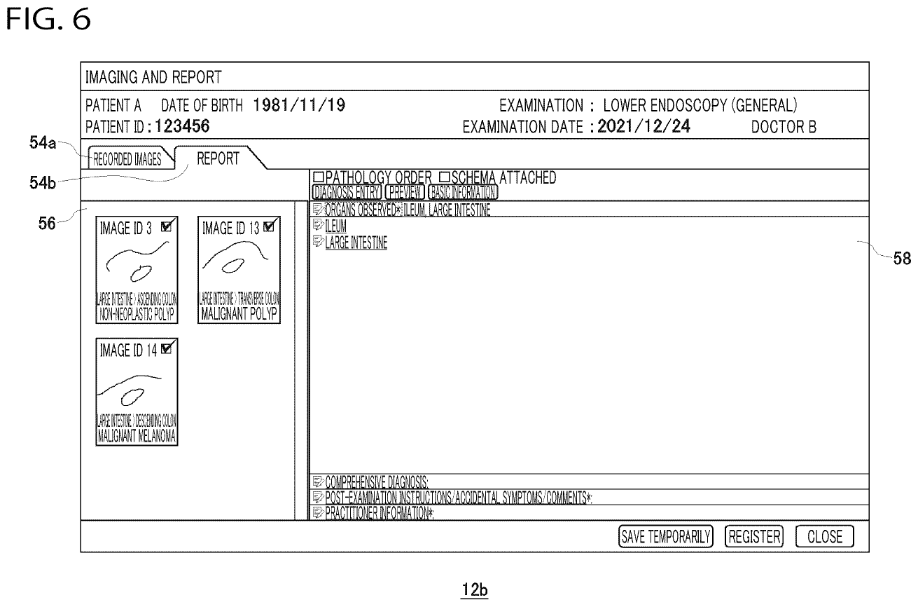

FIG. 6 shows an example of a report creation screen for inputting examination results. The report creation screen constitutes a part of the report input screen. Upon the selection of the report tab 54b, the display screen generation unit 100 generates a report creation screen and displays the report creation screen on the display device 12b. The report creation screen includes two regions: an attached image display region 56 for displaying attached images on the left side; and an input region 58 for the user to input the examination results on the right side. In this example, the endoscopic images with the image IDs 3, 13, and 14 are selected as attached images and displayed in the attached image display region 56.

In the second region 52 shown in FIG. 5, an upper limit may be set for the number of computer-captured images that are displayed. Since the computer-captured images displayed in the second region 52 are images automatically captured by the image analysis device 3 and are not those captured by the user, If the number of displayed images is too large, the amount of effort required for the user to make selections is increased. Therefore, when the number of computer-captured images including a lesion not included in the user-captured images exceeds a predetermined first upper limit, the image specifying unit 102 may specify computer-captured images in a number equal to or less than the first upper limit as candidate images for attachment based on a priority order set according to the type of lesion.

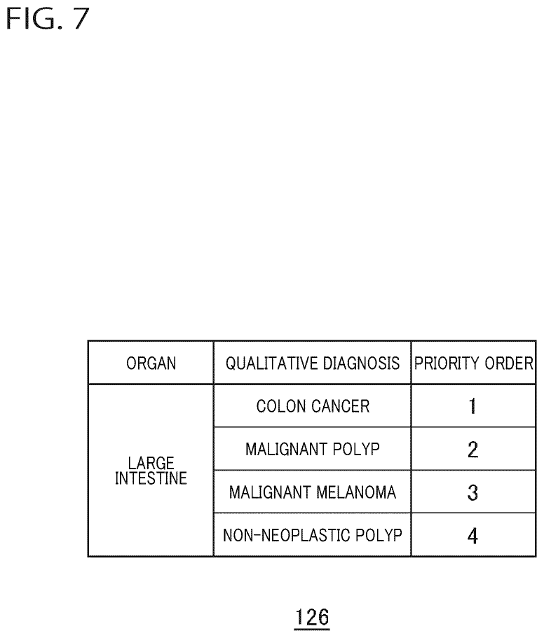

FIG. 7 shows an example of a table stored in the priority memory unit 126. The priority memory unit 126 stores a table that defines the correspondence relationship between a qualitative diagnostic result indicating the type of lesion and the priority order. In this example, with respect to the qualitative diagnosis of the colon (type of lesion), the priority order of colon cancer is given the first priority, the priority order of a malignant polyp is given the second priority, the priority order of malignant melanoma is given the third priority, and the priority order of a non-neoplastic polyp is given the fourth priority. The following explains a case where the upper limit of the number of images to be displayed in the second region 52 is set to three (the first upper limit=3).

In the embodiment, the computer-captured images with the image IDs 8, 10, 13, and 14 are specified as images including a lesion not included in the user-captured images. The qualitative diagnostic result for each computer-captured image is shown below.
Image ID 8: malignant melanoma
Image ID 10: non-neoplastic polyp
Image ID 13: malignant polyp
Image ID 14: malignant melanoma

The image specifying unit 102 specifies three or fewer computer-captured images as candidate images for attachment based on the priority order set according to the type of lesion. In the embodiment, since the number of computer-captured images that include a lesion not included in the user-captured images is four, which exceeds the first upper limit (three), the image specifying unit 102 needs to narrow down the number of computer-captured images to three or less based on the priority order. The priority order for the qualitative diagnostic result for each computer-captured image is shown below.
Image ID 8: malignant melanoma third priority
Image ID 10: non-neoplastic polyp fourth priority
Image ID 13: malignant polyp second priority
Image ID 14: malignant melanoma third priority

Based on the priority order corresponding to the qualitative diagnostic result of each image, the image specifying unit 102 may remove the computer-captured image with the image ID 10 and specify the computer-captured images with the image IDs 8, 13, and 14 as candidate images for attachment. By setting the upper limit for the number of images to be displayed in the second region 52 and selecting the candidate images for attachment based on the priority order set according to the qualitative diagnosis as described, the display screen generation unit 100 can preferentially display the computer-captured images including a significant lesion in the second region 52.

As described above, even when candidate images for attachment are specified based on the priority order, the number of candidate images for attachment may exceed the first upper limit. For example, if the upper limit of the number of images to be displayed in the second region 52 is set to two (the first upper limit=2), since three candidate images for attachment are specified in the above example, the number of the images still exceeds the first upper limit. Therefore, the image specifying unit 102 may further take into account the observation time and select computer-captured images such that the number of candidate images for attachment is the first upper limit or less.

First, the image specifying unit 102 specifies computer-captured images that serve as candidates for candidate images for attachment based on the priority order when the number of computer-captured images including a lesion not included in the user-captured images exceeds the first upper limit (two images). The computer-captured images with the image IDs 8, 13, and 14 are specified as candidates for the candidate images for attachment in this case. When the number of the computer-captured images specified as candidates for the candidate images for attachment (three images) exceeds the first upper limit, the image specifying unit 102 may remove computer-captured images with short user observation time from the candidates for the candidate images for attachment and specify computer-captured images in a number equal to or less than the first upper limit as the candidate images for attachment.

Since the priority order of the image with the image ID 13 is the second and the priority order of the images with the image IDs 8 and 14 are the third, the image specifying unit 102 compares the observation time of the image with the image ID 8 and the observation time of the image with the image ID 14 after determining that the image with the image ID 13 is a candidate image for attachment. The observation time for the image with the image ID 8 is 16 seconds, and the observation time for the image with the image ID 14 is 12 seconds. Since it is predicted that the longer the observation time is, the more the user has been paying attention during the examination, the image specifying unit 102 removes the computer-captured image with the image ID 14, which has the shorter observation time, from the candidate images for attachment and specifies the computer-captured image with the image ID 8 as a candidate image for attachment. Therefore, the image specifying unit 102 may specify the computer-captured images with the image IDs 8 and 13 as candidate images for attachment.

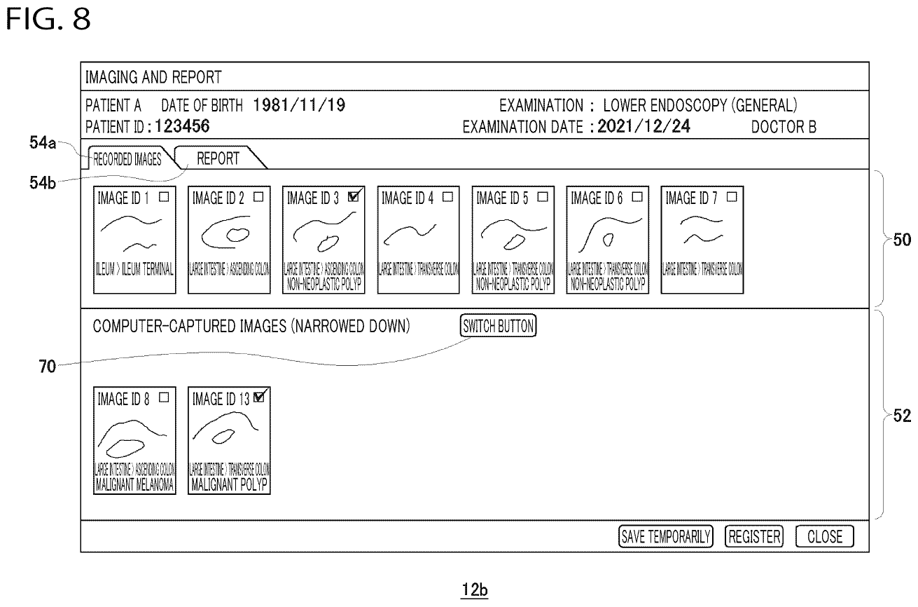

FIG. 8 shows another example of the selection screen for selecting endoscopic images to be attached to a report. Compared to the selection screen shown in FIG. 5, the number of computer-captured images displayed in the second region 52 is limited to two. By limiting the number of images displayed in the second region 52, the user can efficiently select images to be attached to the report. A switch button 70 for displaying all the computer-captured images may be provided in the second region 52.

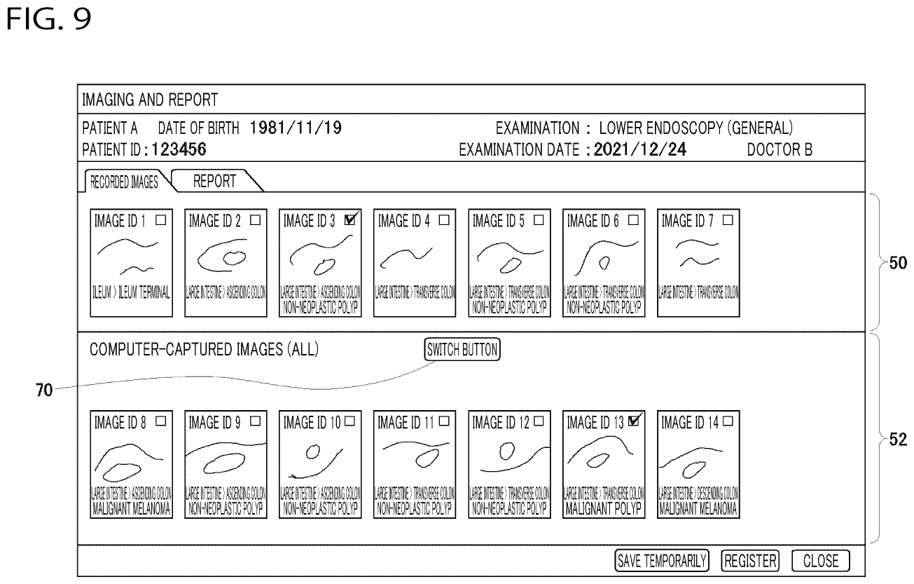

FIG. 9 shows an example of the selection screen when all computer-captured images are displayed. The switch button 70 is used to switch between a mode in which a limited number of computer-captured images are displayed and a mode in which all computer-captured images are displayed. The user can view endoscopic images including all detected lesions by having all the computer-captured images displayed in the second region 52.

The display screen generation unit 100 may display the user-captured images and the candidate images for attachment on the same screen in the first display mode, and display only the candidate images for attachment and not the user-captured images in the second display mode. This mode switching may be implemented by an operation button different from the switch button 70. The display screen generation unit 100 generates selection screens in various modes, allowing the user to select images to be attached to a report from a selection screen that includes captured images that the user desires.

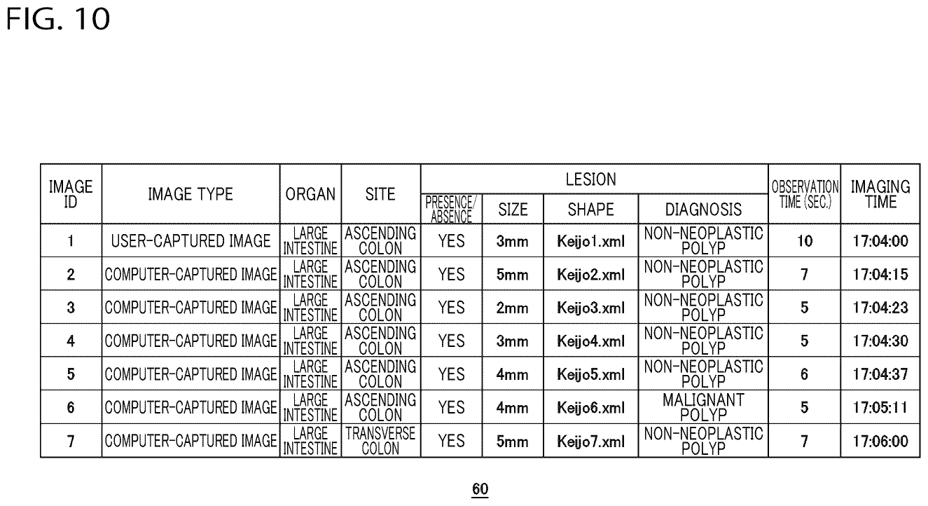

FIG. 10 shows another example of information linked to captured images. In this example, information on the user-captured image with the image ID 1 is stored in the first information memory unit 64, and information on the computer-captured images with the image IDs 2 to 7 is stored in the second information memory unit 66.

In reference to FIG. 10, the user-captured image with the image ID 1 and the computer-captured images with the image IDs 2 to 5 include non-neoplastic polyps in the ascending colon of the large intestine. When the total number of one or more user-captured images and one or more computer-captured images that include a lesion of a predetermined type (a non-neoplastic polyp in the embodiment) in the same site exceeds the predetermined second upper limit, the image specifying unit 102 does not specify the one or more computer-captured images as candidate images for attachment.

When the number of captured images including a non-tumor polyp in the same site exceeds the second upper limit (e.g., four), it is assumed that the user intentionally did not capture the non-tumor polyp. In such a case, since it is not desirable to display an image not captured by the user as a candidate image for attachment, if the total number of user-captured images and computer-captured images that include a lesion of a predetermined type (a non-neoplastic polyp) in the same site exceeds the predetermined second upper limit, the image specifying unit 102 does not select the computer-captured images as candidate images for attachment. In the example of FIG. 10, the image specifying unit 102 preferably specify the computer-captured images with the image IDs 6 and 7 as candidate images for attachment and not specify the computer-captured images with the image IDs 2 to 5 as candidate images for attachment.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure. In the embodiment, the endoscope observation device 5 transmits user-captured images to the image storage device 8. However, in an exemplary variation, the image analysis device 3 may transmit user-captured images to the image storage device 8. In the embodiment, the information processing device 11b has the image specifying unit 102. However, in the exemplary variation, the server device 2 may have the image specifying unit 102.

What is claimed is:

1. A medical support system comprising: one or more processors comprising hardware, wherein
the one or more processors are configured to:
acquire a first image captured through a capture operation by a user and a computer-captured image captured by a computer including a lesion;
specify the computer-captured image including the lesion not included in the first image as a second image; and
generate a selection screen that is for selecting an image to be attached to a report and that includes the first image and the second image.

2. The medical support system according to claim 1, wherein
the one or more processors are configured to:
receive a user's operation of selecting the first image or the second image on the selection screen.

3. The medical support system according to claim 1, wherein
the one or more processors are configured to:
based on information indicating an organ included in the first image and information indicating an organ included in the computer-captured image, determine whether or not there is the first image including the same organ as the organ included in the computer-captured image; and
specify the computer-captured image as the second image when the first image is not present that includes the same organ as the organ included in the computer-captured image.

4. The medical support system according to claim 1, wherein
the one or more processors are configured to:
based on information indicating a site of an organ included in the first image and information indicating a site of an organ included in the computer-captured image, determine whether or not there is the first image including the same site as the site included in the computer-captured image; and
specify the computer-captured image as the second image when the first image is not present that includes the same site as the site included in the computer-captured image.

5. The medical support system according to claim 1, wherein
the one or more processors are configured to:
based on information indicating the size of a lesion included in the first image and information indicating the size of the lesion included in the computer-captured image, determine whether or not there is the first image that includes the lesion whose size is the same as the size of the lesion included in the computer-captured image or whose difference in size from the lesion included in the computer-captured image is within a predetermined range; and
specify the computer-captured image as the second image when the first image is not present that includes the lesion whose size is the same as the size of the lesion included in the computer-captured image or whose difference in size from the lesion included in the computer-captured image is within the predetermined range.

6. The medical support system according to claim 1, wherein
the one or more processors are configured to:
based on information indicating the shape of a lesion included in the first image and information indicating the shape of the lesion included in the computer-captured image, determine whether or not there is the first image that includes the lesion whose shape is the same as the shape of the lesion included in the computer-captured image or whose difference in shape from the lesion included in the computer-captured image is within a predetermined range; and
specify the computer-captured image as the second image when the first image is not present that includes the lesion whose shape is the same as the shape of the lesion included in the computer-captured image or whose difference in shape from the lesion included in the computer-captured image is within the predetermined range.

7. The medical support system according to claim 1, wherein
the one or more processors are configured to:
based on information indicating the type of a lesion included in the first image and information indicating the type of the lesion included in the computer-captured image, determine whether or not there is the first image including the same type of the lesion as the type of the lesion included in the computer-captured image; and
specify the computer-captured image as the second image when the first image is not present that includes the same type of the lesion as the type of the lesion included in the computer-captured image.

8. The medical support system according to claim 1, wherein
the one or more processors are configured to:
determine whether or not there is the first image that include an organ, a site, and a lesion that are the same as those in the computer-captured image based on information indicating an organ, information indicating a site, information indicating the size of a lesion, information indicating the shape of the lesion, and information indicating the type of the lesion that are included in the first image and on information indicating an organ, information indicating a site, information indicating the size of the lesion, information indicating the shape of the lesion, and information indicating the type of the lesion that are included in the computer-captured image; and
specify the computer-captured image as the second image when the first image is not present that includes the same organ, site, and lesion as those included in the computer-captured image.

9. The medical support system according to claim 1, wherein
the one or more processors are configured to:
when the number of computer-captured images including the lesion not included in the first image exceeds a predetermined first upper limit, specify computer-captured images in a number equal to or less than the first upper limit as the second images based on a priority order set according to the type of lesion.

10. The medical support system according to claim 9, wherein
the one or more processors are configured to:
acquire information indicating time spent by the user in observing the lesion included in the computer-captured images;
when the number of candidates for the second images exceeds the first upper limit even after the computer-captured images serving as the second images are specified based on the priority order, remove computer-captured images with short user observation time from the candidates for the second images and specify computer-captured images in a number equal to or less than the first upper limit as the second images.

11. The medical support system according to claim 1, wherein
the one or more processors are configured to:
when the total number of one or more first images and one or more computer-captured images that include a lesion of a predetermined type in the same site exceeds a predetermined second upper limit, not specify the computer-captured images as the second images.

12. The medical support system according to claim 1, wherein
the one or more processors are configured to:
generate the selection screen in which a first region for displaying the first image and a second region for displaying the second image are provided separately.

13. The medical support system according to claim 12, wherein
the one or more processors are configured to:
display the first image and the second image on the same screen.

14. The medical support system according to claim 1, wherein
the one or more processors are configured to:
display the first image and the second image on the same screen in a first display mode; and
display the second image while not displaying the first image in a second display mode.

15. A report creation support method comprising:
acquiring a first image captured through a capture operation by a user;
acquiring a computer-captured image captured by a computer including a lesion;
specifying the computer-captured image including the lesion not included in the first image as a second image; and
generating a selection screen that is for selecting an image to be attached to a report and that includes the first image and the second image.

16. The report creation support method according to claim 15, comprising:
receiving a user's operation of selecting the first image or the second image on the selection screen.

17. An information processing apparatus comprising: one or more processors comprising hardware, wherein
the one or more processors are configured to:
acquire a first image captured through a capture operation by a user and a computer-captured image captured by a computer including a lesion;
specify the computer-captured image including the lesion not included in the first image as a second image; and
generate a selection screen that is for selecting an image to be attached to a report and that includes the first image and the second image.

18. The information processing apparatus according to claim 17, wherein
the one or more processors are configured to:
receive a user's operation of selecting the first image or the second image on the selection screen.
